(19) United States
(12) Patent Application Publication
(10) Pub. No.: US 2024/0032867 A1
(43) Pub. Date: Feb. 1, 2024

(54) WIRELESS IMPLANTABLE SENSING DEVICES

(71) Applicant: THE BOARD OF TRUSTEES OF THE LELAND STANFORD JUNIOR UNIVERSITY, Stanford, CA (US)

(72) Inventors: Ada Shuk Yan Poon, Redwood City, CA (US); Bob S. Hu, Los Altos Hills, CA (US); Jihoon Jang, Stanford, CA (US); Anatoly Yakovlev, Mountain View, CA (US); Yuji Tanabe, Stanford, CA (US); Alex Yeh, Palo Alto, CA (US); Stephanie Hsu, Palo Alto, CA (US); Andrew Ma, Palo Alto, CA (US)

(21) Appl. No.: 18/112,297

(22) Filed: Feb. 21, 2023

Related U.S. Application Data
(63) Continuation of application No. 16/505,425, filed on Jul. 8, 2019, now abandoned.
Application No. 16/505,425 is a continuation of application No. 14/424,303, filed on Feb. 26, 2015, now abandoned.
(60) Provisional application No. 61/720,827, filed on Oct. 31, 2012.

(51) Int. Cl. A61B 5/00; A61B 5/03; A61B 5/0205; A61N 1/05; A61N 5/06; A61N 7/00; A61N 2/00; A61B 5/07; H01L 23/00; A61B 5/145; A61B 5/24

(52) U.S. Cl. CPC A61B 5/686; A61B 5/03; A61B 5/02055; A61N 1/05; A61N 5/0601; A61N 7/00; A61N 2/00; A61B 5/4839; A61B 5/076; A61B 5/0031; H01L 24/48; A61B 5/6869; A61B 5/14503; A61B 5/6876; A61B 5/6868; A61B 5/24; A61B 5/026

(57) ABSTRACT

An implantable device is provided that can include any number of features. In some embodiments, the device includes a coil antenna configured to receive wireless power from a power source external to the patient. The device can include at least one sensor configured to sense a bodily parameter of the patient. The device can also include electronics configured to communicate the sensed bodily parameter of to a device located external to the patient. Methods of use are also described.

### CROSS REFERENCE TO RELATED APPLICATIONS

This application is a continuation application that claims the benefit and priority to the U.S. application Ser. No. 16/505,425 filed Jul. 8, 2019, titled “Wireless Implantable Sensing Devices”, which claims the benefit and priority to the U.S. application Ser. No. 14/424,303, filed Feb. 26, 2015, titled “Wireless Implantable Sensing Devices”, which claims the benefit and priority, under 35 USC 371, to the International Application No. PCT/US2013/067882, filed Oct. 31, 2013, which claims the benefit and priority, under 35 USC 119, to the U.S. Provisional Application No. 61/720,827, filed Oct. 31, 2012, titled “Wireless Implantable Sensing Devices”, which are incorporated herein by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure relates generally to implantable monitoring and sensing devices. More specifically, this disclosure relates to implantable, untethered, wireless monitoring and sensing devices for diagnostic purposes, and devices capable of control for therapeutic purposes.

### BACKGROUND

Cardiac arrhythmias affect more than 5 million people nationwide, and result in more than 1.2 million hospitalizations and 400,000 deaths each year in the United States. Atrial fibrillation (AF) and ventricular tachycardia (VT) account for most of the curable episodes if precise 3D mapping of the depolarization pattern is accessible. In the past five decades, various mapping systems have been proposed and developed. They can provide some but not all of the desired properties of an ideal mapping system.

The most common (AF) and the most lethal (VT) electrical disturbances of the heart are both caused by altered electrical conduction patterns. Therefore, the 3D mapping of the depolarization pattern has been an area of research for more than 5 decades. While conventional electrocardiogram (ECG) provides some ability to localize the pattern of depolarization, a more precise method would be highly desirable.

Methods like magnetocardiography (MCG) require highly specialized equipment and biomagnetic inversion problem is inherently ill-posed mathematically. MCG is a non-invasive mapping technique. But it requires the use of highly sensitive SQUID detector and suffers from sensitivity to noise due to the ill-posed inversion problem. Noninvasive techniques such as MCG have therefore been found unreliable for even investigative use.

Invasive intracardiac mapping is a laborious point-by-point mapping procedure that provides the only reliable analysis of the propagation electrical wavefront. Catheter-based activation and pacing in conjunction with surface electrocardiography and x-ray fluoroscopy is the most commonly used mapping technique in a clinical setting. However, the limitations of this technique are threefold. First, arrhythmia induction is often necessary for precise mapping. Patients who have structural heart disease (SHD) often have poor hemodynamic tolerance to the induced arrhythmia. Second, sequential recording is performed by a single or few electrode catheters maneuvered in the heart chamber. There is an implicit assumption that activations repeat in the same way from cycle to cycle at each site. This might not be a valid assumption in complex rhythms such as polymorphic arrhythmia. Thus, the procedure could last for several hours. Third, the mapping itself is limited to the reachable endocardial surface of the heart. Thus, local electrograms are not true representations of the depolarization pattern, especially in the thicker myocardium of the ventricles. Ideally, high-density maps of cardiac depolarization can be obtained without prolonged mapping.

Electrodes and stents have been implanted in human hearts for several decades. They have been deployed in the cardiac chambers as well as cardiac venous and arterial structures. Electrodes are currently millimeters in size and usually require a direct wired connection for operation. Stents measuring 50-100 microns in thickness are deployed routinely in the coronary arteries but lack of the ability to report back information from the local environment.

In the past few decades, newer mapping techniques have been proposed and developed. CARTO is the first 3D mapping system in electrophysiology (EP) testing. It utilizes a magnetic field sensor incorporated in the tip of the mapping catheter and an external magnetic field emitter located under the patient beneath the operating table. An electroanatomical map is generated when the mapping catheter is maneuvered in the heart chamber. The CARTO system, however, shares the same limitation of sequential recording and hence incurs long procedural time.

Non-contact multi-electrode mapping entails simultaneously recording of electrical activity at multiple sites. Other companies have developed a multi-electrode array with up to 64 electrodes in the shape of a balloon for endocardial mapping. Because the electrodes do not touch the endocardium, the mapping accuracy is limited.

In the UnEmap system developed by a group of researches in the University of Auckland, an epicardial electrode sock with over 100 electrodes is fitted over the heart. It is used extensively in experiments to better understand the underlying mechanisms of cardiac arrhythmias, but is seldom used in the clinical setting due to the invasiveness.

Optical mapping technique advances our understanding of cardiac electrophysiology in ways that have not been accomplished by other approaches. This technique uses a voltage-sensitive dye, invented by Nobel laureate Roger Tsien, to translate voltage changes into an optical signal, and provides better temporal and spatial resolution than other mapping techniques. Additionally, it allows simultaneous recording of membrane potential in the whole heart. Developed on experimental preparations using various species, these optical mapping techniques have recently been applied to the ex vivo human heart. These studies lead to the explanation of ventricular excitation and arrhythmias in terms of the hidden spatio-temporal patterns of propagation within the ventricular wall. However, the voltage-sensitive dyes are toxic. Therefore, optical mapping is not suitable for clinical use.

Table 1 summarizes the properties and limitations of the newer mapping techniques as compared with the conventional catheter-based technique and the invention disclosed herein.

TABLE 1

| | Catheter | CART | Endocardial | Epicardial | | Optical | invention |
|---|---|---|---|---|---|---|---|
| | -based | O | multielectrode | multielectrode | MCG | mapping | This |
| Parallel recording | No | No | Yes | Yes | Yes | Yes | Yes |
| Minimally invasive | Yes | Yes | Yes | No | Yes | No | Yes |
| Non-toxic | Yes | Yes | Yes | Yes | Yes | No | Yes |
| Simple instruments | Yes | Yes | Yes | Yes | No | Yes | Yes |
| In contact with tissue | Yes | Yes | No | Yes | N/A | N/A | Yes |
| True intramyocardial | No | No | No | No | No | No | Yes |
| recording | | | | | | | |

### SUMMARY OF THE DISCLOSURE

A patient monitoring system is provided, comprising a plurality of implantable, wirelessly powered sensing devices, each sensing device comprising an antenna configured to receive wireless power, at least one sensor configured to sense a bodily parameter of the patient, and electronics coupled to the antenna and the sensor and configured to communicate the sensed bodily parameter, and at least one external device configured to provide wireless power to the antennae of the sensing devices and configured to receive the sensed bodily parameters from the sensing devices.

In some embodiments, the at least sensor is selected from the group consisting of electrical sensor, pressure sensor, optical sensor, mechanical sensor, and temperature sensor.

In another embodiment, each sensing device measures less than 1 mm×1 mm×1 mm in size.

In some embodiments, each antenna comprises a 3D coil.

In one embodiment, each of the sensing devices comprises an energy harvesting mechanism that is selected from the group consisting of magnetic harvesting mechanism, optical harvesting mechanism, mechanical harvesting mechanism, thermal harvesting mechanism, and chemical harvesting mechanism.

In some embodiments, the system further comprises a therapy element configured to apply therapy to the patient. In some embodiments, the therapy element is selected from the group consisting of electrode, optical element, ultrasound transducer, chemical element, and magnetic element.

An implantable, untethered sensing device is provided, the untethered sensing device comprising an antenna configured to receive wireless power from a power source external to a patient, at least one sensor configured to sense a bodily parameter of the patient, and electronics coupled to the antenna and the at least one sensor and configured to communicate the sensed bodily parameter to a device external to the patient.

In one embodiment, the at least sensor is selected from the group consisting of electrical sensor, pressure sensor, optical sensor, mechanical sensor, and temperature sensor.

In some embodiments, the device measures less than 1 mm×1 mm×1 mm in size.

In another embodiment, the antenna comprises a 3D coil.

In some embodiments, each of the sensing devices comprises an energy harvesting mechanism that is selected from the group consisting of magnetic harvesting mechanism, optical harvesting mechanism, mechanical harvesting mechanism, thermal harvesting mechanism, and chemical harvesting mechanism.

In one embodiment, the sensing device further comprises a therapy element configured to apply therapy to the patient. In some embodiments, the therapy element is selected from the group consisting of electrode, optical element, ultrasound transducer, chemical element, and magnetic element.

A method of monitoring a patient parameter is also provided, comprising implanting at least one wirelessly powered sensing device into a patient, providing wireless power to the at least one sensing device with an external device, sensing a bodily parameter of the patient with the at least one sensing device, and communicating the sensed bodily parameter from the at least one sensing device to the external device.

In some embodiments, the at least one sensing device is implanted into a part of the body selected from the group consisting of a heart, brain, vascular system, and abdomen of the patient.

In another embodiment, the bodily parameter is selected from the group consisting of an EEG, EKG, ECG, blood pressure, core temperature, blood flow, resistance, impedance, and pressure of fluid within the patient.

In some embodiments, the method further comprises providing therapy to the patient with the sensing device. In some embodiments, the therapy comprises electrical stimulation, chemical stimulation, optical stimulation, or magnetic stimulation, or mechanical stimulation.

In some embodiments of the systems and methods disclosed herein, the sensing devices do not comprise a battery. In some embodiments, the sensing devices are powered only when receiving wireless power from the external device.

In some embodiments, the sensing devices receive wireless power in the mid-field where energy is exchanged through a combination of inductive and radiative modes.

In one embodiment, the sensing devices receive wireless power at an operating frequency ranging from approximately 100 MHz to 5 GHz.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C illustrate one embodiment of an implantable wireless sensing system.

FIG. 2 is a schematic drawing of a system comprising a sensing device and an external device.

FIGS. 3A-3C provide additional views of the 3D structure of the sensing device of FIG. 1C.

FIG. 4 shows another embodiment of a sensing device.

### DETAILED DESCRIPTION

This disclosure describes methods and apparatus for replacing large “dumb” and tethered electrode sensing devices with multiple implantable or injectable “smart” untethered wireless-powered sensing devices. These untethered sensors can include electronics comprising integrated circuit (IC) chips configured to sense body parameters (e.g., an electrogram), a wireless interface to transmit the sensed body parameters to an external device or detector, and a unique identification to locate each sensor. The systems described herein can include control over the types of body parameters to monitor, the duration of monitoring, and the number of locations within the body to monitor simultaneously.

This disclosure provides a novel mapping system comprising a plurality of implantable, wirelessly powered sensing and control devices configured to create a high-density map in real time or on demand of sensed body parameters (e.g., cardiac depolarization) using simple and minimally invasive procedures and sensing devices without the need for prolonged mapping time.

An array of untethered, wirelessly-powered, small, and individually addressable electrode sensing devices can be implanted or injected into the body, such as into the circulatory system or into an organ, muscle, skin or body cavity of the patient. In a cardiac application, these sensing devices can be configured to detect the local depolarization patterns which can then be simultaneously interrogated by an external detector. The external detector can be adapted to demultiplex signals from these sensing devices at different locations, and reconstruct the depolarization map in real time. This mapping system can revolutionize the way of measuring bodily parameters such intracardiac electrical activities, assist cardiologists to ablate complicated arrhythmias, and reduce the procedural time of electrophysiology (EP) testing. In addition, it can provide medical researches a flexible tool to better understand the electrical signal propagation and test out new hypothesis of the initiation of arrhythmias in cardiac tissue.

Wireless sensing and/or therapy devices disclosed herein can integrate the various circuit components of a wireless sensing device (e.g., wireless powering circuits, telemetry circuits, and the electrode sensors) into a single sensor IC chip. The sensing devices disclosed herein can include optimized designs for the implanted antenna, electrode configurations, 3D packaging of the entire probe, and the external detector. It is a goal of the systems described herein to minimize the effect of biological reaction to the presence of implanted wireless sensing devices.

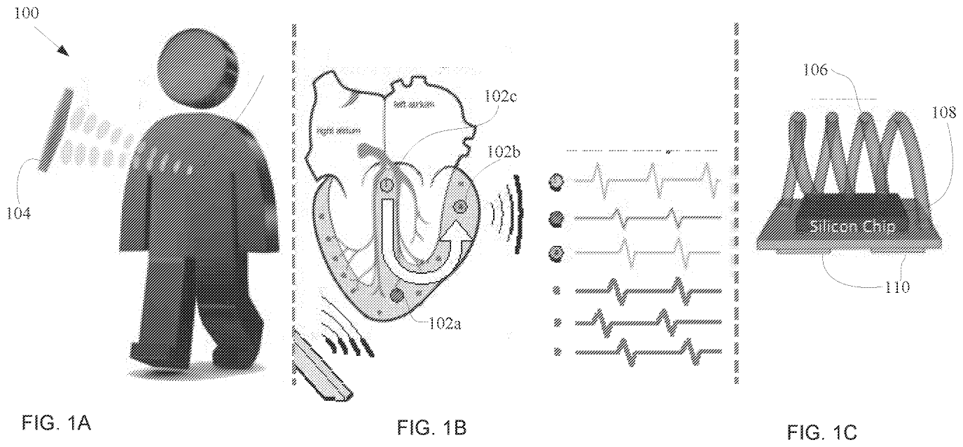

FIGS. 1A-1C illustrate one embodiment of a wireless sensing system 100. Referring to FIG. 1C, the system 100 can include, for example, at least one implantable sensing device 102 configured to receive wireless power, and an external wireless power source and detector 104 configured to transmit and/or receive wireless power and communications. The wireless power source can include, for example, a transmitter coil connected to a power source. In some embodiments, the wireless power source can be separate from the detector, and in other embodiments they can be integrated into the same device. Any number of sensing devices can be implanted in the human body, depending on the bodily parameters to be sensed. For example, several sensing devices can be implanted on or within the heart of a patient to map local depolarization patterns of the heart.

As will be described in more detail below, the sensing devices can be untethered, wirelessly powered sensing devices configured to sense a body parameter of a patient and wirelessly communicate the sensed information to an external device. In some embodiments, the sensing devices can be configured to sense one or more of the following: parameters of the body, such as ECG, EKG, EEG, resistance, or impedance, pressure parameters of the body such as the pressure of fluids within a lumen or an organ, temperature of the body or of bodily fluids, or optical parameters of the body, glucose content, or other chemical, biological, or particular molecular content in the blood or other organs. In some cases, certain composition of material can be sensed, such as presence of blood, puss, or bacterial infection. This can be achieved by including appropriate sensors on the sensing devices, such as electrodes, pressure sensors, temperature sensors, optical sensors, etc.

In one embodiment, a plurality of implanted sensing devices forms a network of sensing and stimulating devices that can operate in coordinated manner to close the loop for action with respect to measured quantities. For instance, in one embodiment, a device could measure cardiac output such as blood flow out of heart and another device could stimulate the heart to regulate the cardiac output.

FIG. 1B shows multiple sensing devices 102a, 102b, and 102c implanted at different locations in or on the heart. In some embodiments, the sensing devices can be implanted or injected directly into the myocardium of the heart, or alternatively, can be affixed to the epicardium or endocardium of the heart. FIG. 1B also shows various electrogram readings measured by the sensing devices. Although FIG. 1B shows the sensing devices being implanted in the heart, it should be understood that in other embodiments, the sensing devices can be implanted in other parts of the body.

In some embodiments, when the sensing devices are implanted within the heart of a patient, as shown in FIG. 1B, each sensing device can measure an electrical parameter such as the local electrogram. The external device or detector of the system can be configured to interrogate the measurement from each sensing device and deduce the propagation of excitation wavefront versus time. In some embodiments, the complete interrogation period for all sensing devices can be short enough compared to the cardiac signals in order to achieve high temporal resolution. Multiple sensing devices can be deployed within the body to simultaneously measure electrograms or other bodily parameters. Due to miniature form factor of the sensors, the numerous implanted devices can also achieve high spatial resolution.

FIG. 1C illustrates one embodiment of an implantable sensing device 102. The sensing device 102 can be delivered via injection, for example, and can comprise an antenna 106, an integrated-circuit (IC) chip 108, and at least one sensor 110. In some embodiments, the antenna 106 can comprise a coil connected to the IC chip 108 to form a resonant or non-resonant system. The antenna 106 of the sensing device 102 can be inductively coupled to the wireless power source and detector 104 of FIG. 1A to receive wireless power and communications, and also to transmit communications to the detector. In some embodiments, the device size can be 1×1×1 mm, if sufficient power can be transferred to the device. In other embodiments, the device can be elongated in one dimension such that its diameter is small enough to fit in the needle, but has a larger antenna cross section for higher power harvesting capability. Depending on the operating environment, delivery method, and power consumption, a device could be as little as 100 μm and up to several centimeters in one or more dimensions.

In some embodiments, the sensor 102 can comprise an electrode, but it should be noted that in other embodiments the sensor can be any type of sensor adapted to measure a bodily parameter, such as a pressure sensor, optical sensor, temperature sensor, etc. The sensing device can optionally include other features, such as a power source (e.g., a battery, a capacitor, etc.). In some embodiments, each sensing device can include a unique identification (ID) so as to identify the individual sensors. The unique ID can be used by the IC chip 108, or by an external detector (such as external detector 104) to identify the measurements taken by that individual sensor or induce localized stimulation to a particular region of the organ.

The IC chip 108 can be configured to provide signal processing directly on the sensing device. In some situations, it can be cheaper and more efficient to process the measured data on the sensing device itself, and transmit the processed data to the external device. Processing locally can be more efficient if the processing can be done with fairly simple analog or digital circuitry. The IC chip can include an energy harvesting and power management block, a matching network, a transceiver for telemetry with external reader, a sensor interface with signal conditioning circuitry, a signal conditioning and pulse generation block for stimulation, data conversion circuits, auxiliary circuit blocks, such as power-on-reset circuits, and a controller. Integrating all the electronics described into a single IC chip leads to having fewer discrete components and therefore allows the sensing devices to be miniaturized to the sizes described herein.

The controller can manage the localized device operation, process commands from the external reader, packetize and send out sensed data back to the external reader. The controller can also contain digital or analog signal processing blocks which can analyze and process sensed data and can adjust the device's course of action or performance based on the processed information. For instance, if the controller determines that its analog-to-digital converter resolution is too low, it can increase its effective resolution or adjust gain of signal conditioning circuit to improve the performance, without engagement of the external reader. One other example for adjustment of performance is to automatically tune the cutoff frequencies of the filters in the sensor signal conditioning block in order to pass only the desired frequency components and filter out the interfering signals and noise. This may be done by the IC chip autonomously if all the necessary signal processing components are integrated on chip. Alternatively, the device can send raw data to the external reader. The reader can process the data and adjust the necessary parameters of the IC chip based on the information it receives by sending configuration commands to the IC chip.

Electronics and mechanical systems, empowered by modern CMOS, MEMS, and nanofabrication technologies, have been miniaturized faster than electro-chemical energy storage. As a result, embedded batteries typically dominate the size and weight of implanted medical devices. To combat this, in some embodiments the sensing devices of the present disclosure can be powered externally by the external power source and detector via transcutaneous wireless power transfer. The external detector 102 can include a transmit coil that can be coupled to a receive coil on each sensing device. The receive coil can be, for example, the antenna 106 illustrated in FIG. 1C.

The transmit coil of the external detector and the receive coil of the sensing device can form a coupled circuit, whereby current flowing in the transmit coil can create a magnetic field which induces current to flow in the receive coil of the one or more sensing devices. This induced current can then be used to power the sensing devices. In some embodiments, the sensing devices can be configured to operate and measure bodily parameters only when they are in the presence of a magnetic field formed by the external device. Thus, the sensing device 102 can be miniaturized by not including a battery or energy source. When the antenna of the sensing device is in the presence of the magnetic field formed by the external device, the sensing device can turn on or “wake up” and collect or information relating to body parameters. The sensing device can also communicate this sensed information externally while receiving wireless power.

Traditional wireless power transfer across human tissue operates in the near-field, where the transmit and receive coils include inductively coupled coils. Recently proposed systems for mid-range power transfer over air and through tissue also occur in the near-field; high efficiency can be obtained by tuning identical resonators to operate in the strongly coupled regime. Power transfer to medical implants, however, typically operates in the weakly coupled regime due to the asymmetry between a large external transmit coil and the small receive coil on the implant. In this configuration, it has been shown that optimal power transfer occurs in the mid-field where energy is exchanged through a combination of inductive and radiative modes.

Typical implantable devices rely on inductive coupling to harvest power and communicate with the external transmitter. This often implies large coil antennas for both the external transmitter and the implant device antenna. One of the key disadvantages of the inductive coupling is that these antennas cannot be significantly miniaturized because they become very inefficient. Increasing the frequency of operation increases the tissue absorption and tissue heating, however, it also increases antenna efficiency for very small antennas. Therefore, there is an optimal frequency at which enough power can be delivered to the small antennas, while limiting tissue heating to safe levels. In some embodiments, an optimal frequency of operation for the implantable sensors disclosed herein can be from approximately 100 MHz up to 5 GHz, depending on implantable device design, operating environment, required power and voltage, and device size.

Operating the implantable sensing devices of this disclosure for mid-range power transfer over high operating frequencies allows for miniaturization of the sensing devices that would not be possible in a system that uses inductive coupling. For example, sensing devices of the present disclosure can be miniaturized to have a total volume on the order of 1 mm×1 mm×1 mm. Another benefit of higher frequency of operation is the higher available bandwidth that can be used to achieve higher data rates for communication. Also, higher frequency of operation desensitizes power transfer efficiency to alignment and orientation between external and implant device antennas.

Wirelessly powering the sensing devices of the present disclosure eliminates the need for leads extending through the skin, which simplifies the implantation procedure and reduces the risk of infection. The sensing devices described herein are therefore less invasive, and safer in the long term for a patient. This is especially important in applications such as brain or cardiac monitoring, where the risk of infection is so great. This also enables longer-lasting implantable devices by eliminating the need for a patient to undergo another surgery to replace a battery. This can be achieved by recharging a battery or energy storage element.

Although it can be advantageous to eliminate the size and weight of conventional chemical batteries, in some embodiments the sensing devices can include an energy source, such as a battery or a capacitor, which can be configured to store energy from the external device and power the sensing devices even when the external device is not wirelessly transmitting power to the sensing devices. This type of configuration can allow the sensing devices to monitor the patient even in the absence of an external charging device.

The sensing devices of FIGS. 1A-1C can also be configured for wireless communication of data, to communicate the measured body parameters outside of the body. This data can be communicated to the external device via a wireless chip incorporated into the IC chip 108, or alternatively, the data can be modulated onto the wireless power transfer signals between the transmission and receive coils of the external device and sensing devices, respectively. In some embodiments, the wireless communication of data can occur only during wireless power transfer between the external device and the implanted sensing devices, so as to reduce power consumption of the device during normal operation.

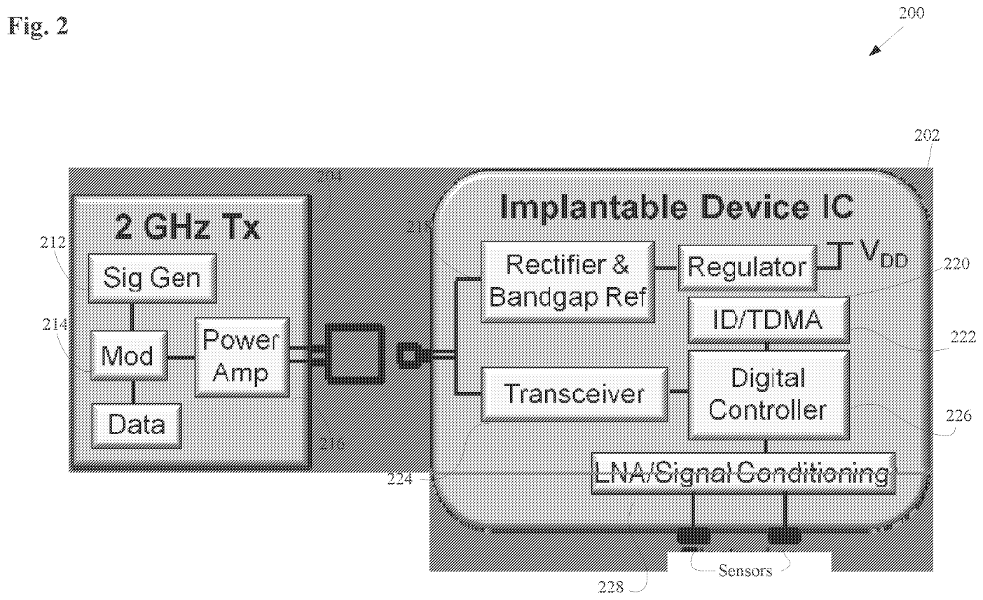

FIG. 2 is a schematic drawing of a system 200 comprising sensing device 202 and external device 204. The sensing device 202 and external device 204 can correspond to sensing device 102 and external device 104 of FIGS. 1A-1C. The external device can wirelessly transfer power and data with a signal generator 212, a frequency modulator 214, and one or more power amplifiers 216. In FIG. 2, the individual components of the sensing device (e.g., rectifier and bandgap ref 218, regulator 220, ID/TDMA 222, transceiver 224, digital controller 226, LNA/signal conditioning module 228, etc.) can be incorporated into the IC chip shown above in FIG. 1C.

Referring to FIG. 2, as recorded signals from the sensors (e.g., electrodes) are corrupted by motion artifact, dc-offset due to the skin-electrode contact resistance, and the 60-Hz interference, the sensor frontend LNA/signal conditioning module 238 can process the measured extracellular signal to obtain a clean signal and detect the timing of local depolarization. All the handshaking protocols with the external device, for example, the multiple access protocol, and the coordination among various building blocks within the cardiac probe can be coordinated by the digital controller 226. The ID of individual probe can be stored in the ID block 222. The transceiver block 224 modulates the processed intracellular signals and sends it to the external device, and demodulates received signals, for example, commands, from the external device. The rectifier 218 and regulator blocks 220 convert the oscillating radio waves incident on the received antenna to dc power for the operation of the sensing device.

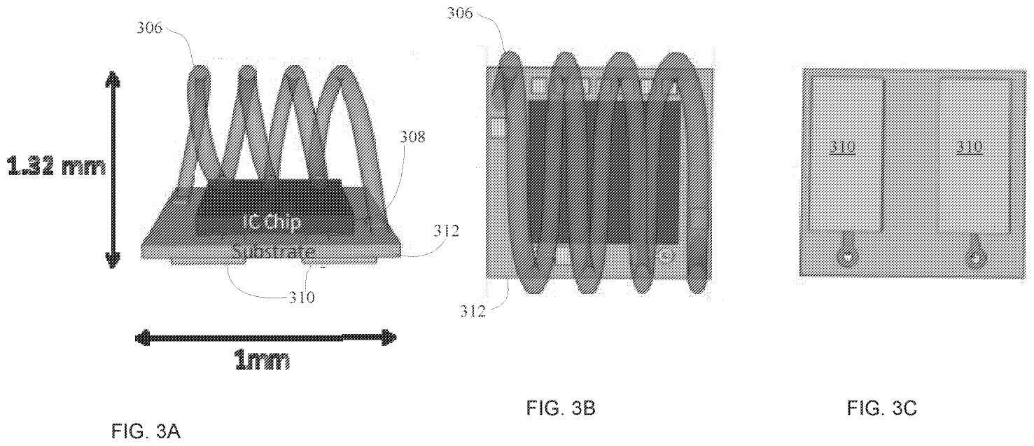

FIGS. 3A-3C provide additional details on the 3D structure of one embodiment of the sensing devices (expanding on what is shown in FIG. 1C). Since it is desirable to have the entire implanted device be as small as possible, this embodiment adopts a 3D packaging approach. The implanted antenna 306 can be a multi-turn micro-coil, for example. The antenna can be wire-bonded to the pads on a supporting substrate 312. The IC chip 308 and sensors 310 can also be bonded to the substrate. FIG. 3B shows a top-down view of the sensing device, and FIG. 3C shows a bottom-up view of the sensing device, giving a better view of sensors 310. In some embodiments, the sensing devices of FIGS. 3A-3C can be approximately 1 mm wide, 1 mm long, and 1.3 mm tall. In another embodiment, the sensing device can be 1 mm×1 mm×1 mm. In another possible embodiment, the device can be encapsulated in an optically transparent package that would enable optical methods of energy harvesting, stimulation, and sensing. Other types of transparency for packaging can be extended to include RF transparent materials, etc.

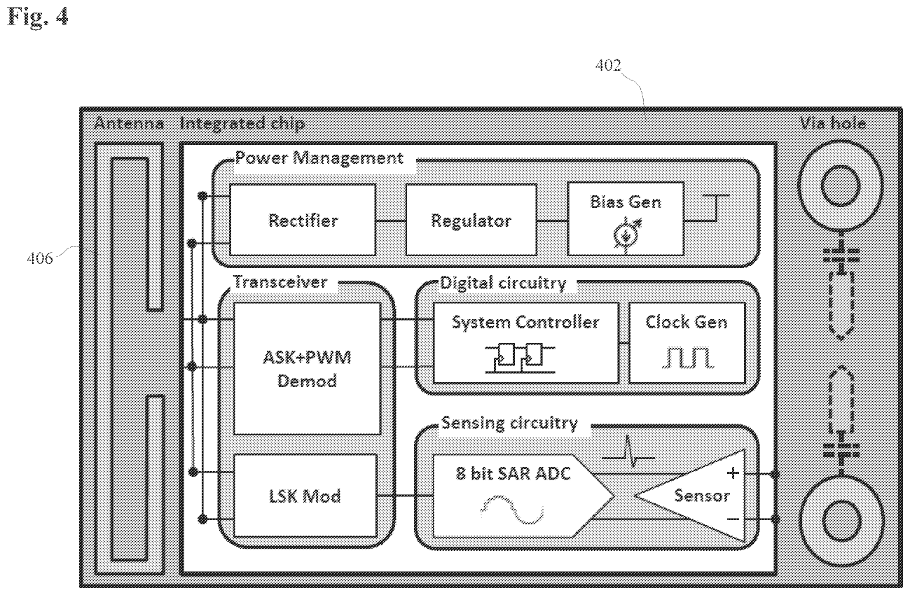

FIG. 4 shows another embodiment of a sensing device 402. In this embodiment, a planar loop antenna 406 can be used for the implantable device. The tradeoff between multi-turn antenna in the form of 3D coil versus a planar antenna with the same diameter, is that the 3D coil antenna has higher induced voltage, which can be advantageous to improve rectifier efficiency. However, it has additional losses associated with it and, therefore, can harvest less power as compared to a planar loop antenna. The illustrated components of the IC chip in FIG. 4 can correspond to the IC chip components described above in FIG. 2.

Compact, wirelessly powered, untethered sensing devices such as those described herein can be used in any number of medical applications. As described above, an array of sensing devices can be used to measure and map the electrical properties of the heart, and communicate that information wirelessly outside of the body to an external device. Uses within the heart are not limited to electrical properties, however, and the sensing devices can also be used to monitor blood flow, heart rate, core temperature, and more.

In another embodiment, a plurality of sensing devices outfitted with pressure sensors can be implanted within the venous system of a patient (e.g., within the pulmonary arteries) to measure the pressure of blood or the flow rate of blood at various points in the venous system.

In another embodiment, a plurality of sensing devices outfitted with electrical sensors or electrodes can be disposed on or near coronary stents and be configured to measure a resistance of blood inside the coronary stents as a way to monitor the degree of stenosis inside the stent. In this embodiment, the sensing devices could be used to report when a stent is wearing down and due for replacement. Thus, the sensing devices of the present disclosure can be used to monitor the effectiveness and lifetime of other medical devices implanted in the body.

The sensing devices described herein can also be implanted on or within the brain to measure various parameters relating to electrical activity, blood flow, or pressure in the brain. For example, in some situations surgeons need info on the local perfusion of the brain, since swelling after surgery can compromise that part of the brain. In this particular embodiment, sensing devices can be implanted in the brain to measure blood flow or other brain parameters as a way of monitoring the patient after surgery. The sensing devices described herein allow for measurement of brain activity directly without having leads that extend out through the skull, thereby reducing infection risk.

Until this point, the sensing devices have been described entirely as sensing or measurement devices. However, in some embodiments, the sensing devices can also include a therapy element, such as a stimulation electrode, configured to provide therapy to a patient. For example, the sensors 110 of FIG. 1C can comprise electrodes and can be configured to provide, for example, cardiac or deep brain stimulation. In other embodiments, the therapy element can be configured to provide magnetic, electrical, optical, ultrasound, or chemical stimulation to bodily tissue or fluids.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. (canceled)

2. A sensing system comprising:
an external controller device; and
a plurality of implantable untethered sensing devices, wherein at least one implantable untethered sensing device of the plurality of implantable untethered sensing devices comprises:
an antenna configured to receive mid-field wireless power from the external controller device at an operating frequency between 100 MHz and 5 Ghz;
at least one sensor configured to sense a body parameter of a patient; and
electronics comprising an energy harvesting and power management block configured to harvest energy and manage power within the electronics, and wherein the electronics further comprises a digital controller coupled to the antenna and the at least one sensor,
wherein the digital controller is configured to process commands from the external controller device to adjust performance of the at least one implantable untethered sensing device,
wherein the digital controller is further configured to process sensed data collected by the at least one sensor,
wherein the digital controller is further configured to adjust one or more parameters associated with the at least one implantable untethered sensing device to adjust performance of the at least one implantable untethered sensing device,
wherein the at least one implantable untethered sensing device implanted on or within the brain of the patient,
wherein the at least one sensor is configured to sense electrical activity of the brain, and
wherein the external controller is configured to recover the sensed electrical activity from the at least one implantable untethered sensing device in real-time.

3. The sensing system of claim 2, wherein the energy harvesting and power management block is one of a magnetic harvesting mechanism, an optical harvesting mechanism, a mechanical harvesting mechanism, a thermal harvesting mechanism, or a chemical harvesting mechanism.

4. The sensing system of claim 2, wherein the at least one implantable untethered sensing device includes a therapy element configured to apply a therapy to the patient.

5. The sensing system of claim 4, wherein the therapy element is one of an electrode, an optical element, an ultrasound transducer, a chemical element, or a magnetic element.

6. The sensing system of claim 4, wherein the therapy comprises deep brain stimulation.

7. The sensing system of claim 2, wherein the at least one implantable untethered implantable sensing device does not comprise a battery.

8. The sensing system of claim 2, wherein the at least one implantable untethered sensing device is powered only responsive to receiving wireless power from the external controller device.

9. The sensing system of claim 2, wherein the sensed electrical activity comprises EEG.

10. The sensing system of claim 2, wherein the antenna comprises a planar loop.

11. The sensing system of claim 2, wherein the antenna comprises a multi-turn coil wire-bonded to a substrate.

12. The sensing system of claim 2, wherein the at least one implantable untethered sensing device is contained within a 1 mm×1 mm×1 mm volume.
